(19) United States
(12) Patent Application Publication
(10) Pub. No.: US 2026/0166234 A1
(43) Pub. Date: Jun. 18, 2026

(54) AEROSOL SIZE CONTROL CORE AND AEROSOL SIZE CONTROL NEBULIZATION DEVICE

(71) Applicant: JTM CO., LTD., Taoyuan City (TW)

(72) Inventors: YU-CHUNG HSU, Taoyuan City (TW); CHIEN-HUA LIN, Taoyuan City (TW); KAI-YAO LO, Taoyuan City (TW); HSIN-HUA TSENG, Taoyuan City (TW)

(73) Assignee: JTM CO., LTD., Taoyuan City (TW)

(21) Appl. No.: 19/370,808

(22) Filed: Oct. 28, 2025

Related U.S. Application Data
(60) Provisional application No. 63/713,598, filed on Oct. 30, 2024.

(51) Int. Cl. A61M 11/00; A61M 11/02; A61M 15/00

(52) U.S. Cl. CPC A61M 11/002; A61M 11/003; A61M 11/02; A61M 15/002; A61M 15/0028; A61M 15/0086

(57) ABSTRACT

The present disclosure provides an aerosol size control core configured to match a nebulization unit, and an aerosol size control nebulization device. The aerosol size control core includes: an enclosing unit, configured to enclose an aerosol generated by the nebulization unit, where the enclosing unit defines an internal aerosol space, to form a controlled flow environment. The enclosing unit has at least one aerosol outlet, the at least one aerosol outlet is in communication with the aerosol space, and the at least one aerosol outlet is constructed to direct an aerosol within a preset particle size range in the aerosol space out of the enclosing unit. And the enclosing unit is constructed to: cause a flow direction of a droplet formed by aerosol condensation in the aerosol space to be different from a flow direction of the aerosol directed out of the enclosing unit through the at least one aerosol outlet. The aerosol size control nebulization device includes the nebulization unit and the aerosol size control core. The aerosol size control core and the aerosol size control nebulization device of the present disclosure allow the aerosol flow out after being formed into the preset particle size range, thereby controlling a particle size.

### BACKGROUND OF THE INVENTION

### 1. Field of the Invention

The present disclosure relates to a nebulization device, and in particular, to an aerosol size control core for a nebulization device, and an aerosol size control nebulization device with the foregoing core.

### 2. Description of the Related Art

In a past nebulization device, an aerosol is generated by using an aerosol generating element. Therefore, a particle size of the aerosol is determined when the aerosol is generated. For example, in a micro-mesh nebulization device, the particle size of the aerosol is determined based on a micropore plate, and in a jet nebulization device or an ultrasonic nebulization device, the particle size of the aerosol is determined based on a mode of impact. Such a design causes uneven distribution of the particle size of the aerosol of the existing nebulization device, or causes a cost of controlling the particle size of the aerosol to be very high.

In addition, in the related art, in a process of generating the aerosol, the particle size exists in a normal distribution pattern, resulting in a large overall particle size of the aerosol. Consequently, the device cannot further generate an aerosol having a smaller particle size, and cannot perform more effective treatment that penetrates deep into lungs.

### BRIEF SUMMARY OF THE INVENTION

In order to improve the foregoing problem in the related art, the present disclosure provides an aerosol size control core for a nebulization device, and a nebulization device that has the foregoing aerosol size control core and generates a smaller particle size.

To achieve the foregoing objective and other objectives, the present disclosure provides an aerosol size control core configured to match a nebulization unit. The aerosol size control core includes: an enclosing unit, configured to enclose an aerosol generated by the nebulization unit, where the enclosing unit defines an internal aerosol space, to form a controlled flow environment. The enclosing unit has at least one aerosol outlet, the at least one aerosol outlet is in communication with the aerosol space, and the at least one aerosol outlet is constructed to direct an aerosol within a preset particle size range in the aerosol space out of the enclosing unit. And the enclosing unit is constructed to: cause a flow direction of a droplet formed by aerosol condensation in the aerosol space to be different from a flow direction of the aerosol directed out of the enclosing unit through the at least one aerosol outlet.

Optionally, the at least one aerosol outlet is provided on a top surface, a side surface, or a bottom surface of the enclosing unit.

Optionally, the aerosol size control core further includes a particle size screening structure, where the particle size screening structure is disposed in the aerosol space, and the particle size screening structure is configured to prevent an aerosol with a particle size larger than the preset particle size range from passing through the at least one aerosol outlet.

Optionally, the particle size screening structure includes one of a flow guiding surface, a baffle, or a micro-mesh.

Optionally, the aerosol size control core further includes a liquid guiding portion disposed in the enclosing unit, where the liquid guiding portion includes at least one inclined surface, groove, convex rib, or curved surface, or any combination thereof, and the liquid guiding portion is configured to cause the aerosol condensed in the aerosol space to flow back to a liquid supply area.

Optionally, the enclosing unit has a plurality of aerosol outlets, and a quantity of the particle size screening structures and the liquid guiding portions is the same as a quantity of the plurality of aerosol outlets, to cause the plurality of aerosol outlets, the particle size screening structures, and the liquid guiding portions to be configured in one-to-one correspondence.

The present disclosure further provides a nebulization device that can control a particle size and generate a smaller particle size, including: a nebulization unit and the aerosol size control core described above. The nebulization unit is configured to nebulize a liquid to generate an aerosol. And the aerosol space accommodates the nebulization unit or is adjacent to the nebulization unit, to enclose the aerosol generated by the nebulization unit.

Optionally, the nebulization unit is one of an aerodynamic jet nebulization module, an ultrasonic nebulization module, or a micro-mesh nebulization module.

Optionally, the nebulization unit is the aerodynamic jet nebulization module, an impact element is disposed inside the enclosing unit, and the impact element is constructed to: generate a primary aerosol by being impacted by a high-speed airflow generated by the nebulization unit.

Optionally, the enclosing unit further has a mounting portion, and is configured to be aligned with a nozzle of the nebulization unit. The impact element is disposed in the enclosing unit in a manner of a detachable structure or a fixed structure through the mounting portion.

Optionally, the nozzle of the nebulization unit and the enclosing unit are of an integrally formed structure, and a relative position of the nozzle and the enclosing unit remains fixed.

In this way, the aerosol size control core of the present disclosure causes, by using a structural design inside the core, the aerosol in the core to flow out after being formed into the preset particle size range, causes a droplet that is condensed by an aerosol having a large particle size (not within the preset particle size range) to flow back to the original liquid supply area through the liquid guiding portion, and may lead to droplet re-aerosolization. Through such a design, the particle size can be controlled by incorporating the aerosol size control core of the present disclosure into the nebulization device without changing the original structure of the nebulization device, thereby reducing development time and a cost of the nebulization device. The aerosol size control core of the present disclosure may alternatively be combined with the nebulization unit, to form an aerosol size control nebulization device. In addition, a flow direction of the aerosol within the preset particle size range exiting the aerosol outlet is different from a flow direction of the condensed droplet. In this way, a condition in the related art in which the generated aerosol and the backflow liquid flow out from a same outlet, causing the large droplet to still remain on an aerosol path (such as a medicine cup, a breathing tube, a face mask, or a mouth piece) and fail to be sufficiently nebulized, and causing medicinal solution residue and waste may further be improved.

To further understand the features and technical content of the present disclosure, refer to the following detailed descriptions and accompanying drawings related to the present disclosure. However, the descriptions and the accompanying drawings are merely used to describe the present disclosure, but are not intended to limit the scope of the claims of the present disclosure.

### BRIEF DESCRIPTION OF THE DRAWINGS

FIG. 1 is a three-dimensional schematic diagram of an aerosol size control core according to a first embodiment of the present disclosure;

FIG. 2 is a three-dimensional schematic diagram of FIG. 1 from another perspective;

FIG. 3 is a schematic cross-sectional view of an aerosol size control core according to a first embodiment of the present disclosure;

FIG. 4 is an exploded view of an aerosol size control nebulization device according to a first embodiment of the present disclosure;

FIG. 5 is a three-dimensional schematic diagram of an aerosol size control core according to a second embodiment of the present disclosure;

FIG. 6 is a three-dimensional schematic diagram of an aerosol size control core according to a third embodiment of the present disclosure;

FIG. 7 is a three-dimensional schematic diagram of an aerosol size control core according to a fourth embodiment of the present disclosure;

FIG. 8 is a three-dimensional schematic diagram of an aerosol size control core according to a fifth embodiment of the present disclosure;

FIG. 9 is a three-dimensional schematic diagram of an aerosol size control core according to a sixth embodiment of the present disclosure;

FIG. 10 is a drug deposition distribution diagram of nebulization devices of other brands and a nebulization device according to the present disclosure; and

FIG. 11 is a drug particle size distribution diagram of a nebulization device using an aerosol size control core according to the present disclosure.

### REFERENCE NUMERALS

1 Enclosing unit
11 Top wall
12 Side wall
13 Aerosol outlet
14 Assembly port
15 Mounting portion
16 Through hole
2 Particle size screening structure
3 Liquid guiding portion
4 Nebulization unit
41 Medicine cup
42 Venturi tube
43 Impact element
431 Nozzle outlet
5 Output tube
S Aerosol space

### DETAILED DESCRIPTION OF THE INVENTION

For thorough understanding of the present disclosure, the present disclosure is described in detail through the following specific embodiments and with reference to the accompanying drawings. A person skilled in the art may understand objectives, features, and effects of the present disclosure based on content disclosed in this specification. It should be noted that the present disclosure may be implemented or applied through other different specific embodiments, and details in this specification may also be modified and changed in various ways based on different viewpoints and applications without departing from the idea of the present disclosure. In addition, the accompanying drawings of the present disclosure are merely for brief illustration, and are not depicted based on actual dimensions. The following implementations further describe related technical content of the present disclosure in detail, but the disclosed content is not used to limit the claims of the present disclosure. The descriptions are as follows.

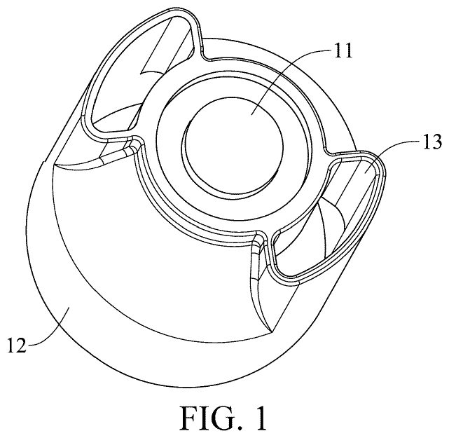

As shown in FIG. 1 to FIG. 4, an aerosol size control core according to a first embodiment of the present disclosure is configured to match a nebulization unit 4, to generate an aerosol within a preset particle size range. The aerosol size control core includes an enclosing unit 1.

The enclosing unit 1 is configured to enclose the aerosol generated by the nebulization unit 4, and the enclosing unit 1 defines an internal aerosol space S, to form a controlled flow environment. Referring to FIG. 1, the enclosing unit 1 is in a form of a housing in this embodiment, and has a top wall 11 and a side wall 12. The top wall 11 and the side wall 12 define the foregoing aerosol space S, to accommodate at least a part of the nebulization unit 4. The nebulization unit 4 may enter from an assembly port 14 of the enclosing unit 1 to be assembled with the enclosing unit 1. However, the present disclosure is not limited thereto, and a specific structure of the enclosing unit 1 may be adjusted based on a requirement. Any element that can enclose the aerosol generated by the nebulization unit 4 and have the aerosol space S may be used as the enclosing unit 1 of the present disclosure.

The enclosing unit 1 has at least one aerosol outlet 13, the at least one aerosol outlet 13 is in communication with the aerosol space S, and the at least one aerosol outlet 13 is constructed to direct the aerosol within the preset particle size range in the aerosol space S out of the enclosing unit 1. Specifically, in this embodiment, the top wall 11 has a plurality of aerosol outlets 13, and allows an aerosol that has a relatively small particle size and falls within the preset particle size range (for example, less than or equal to 3 μm) in the aerosol formed by liquid nebulization to pass upward. Another aerosol that has a relatively large particle size cannot pass through the aerosol outlet 13, and is finally condensed in the aerosol space S and flows back along an inner wall of the enclosing unit 1. In other words, only an aerosol with a particle size is sufficiently small can pass through the aerosol size control core, to jet a nebulized aerosol upward. A value of the preset particle size range may be determined by adjusting parameters such as a size, a shape, a height, a position, a quantity, and distribution of the aerosol outlet 13, thereby enabling only a small-particle-size aerosol that has a good effect and can penetrate deep into a lung of a human body to be directed out of the enclosing unit 1 and enter a deep position of the lung of the human body. A remaining large-particle-size aerosol that does not meet the preset particle size range is condensed into droplets, and the droplets are re-merged and flow back to a liquid supply area, which is usually a medicine cup filled with medicinal solution. These re-merged droplets may continue to be nebulized by the nebulization unit 4, without being adhered to a cup wall of the medicine cup and causing drug residue waste.

The enclosing unit 1 of the present disclosure is further constructed to: enable a flow direction of a droplet formed by aerosol condensation in the aerosol space S to be different from a flow direction of the aerosol directed out of the enclosing unit 1 through the at least one aerosol outlet 13. As shown in FIG. 1 to FIG. 3, because the aerosol within the preset particle size range is light enough, the aerosol can pass upward through the aerosol outlet 13, and the remaining aerosol that does not meet the preset particle size range is condensed into the droplet, and flows back along an inside (for example, the side wall 12) of the enclosing unit 1. The aerosol within the preset particle size range does not follow the same flow direction as that of the condensed droplet. Therefore, a limitation of the particle size is essentially set.

In this embodiment, the aerosol outlet 13 is provided on a top surface (the top wall 11) of the enclosing unit 1, and the condensed droplet flows to the assembly port 14 (where the condensed droplet may exit the enclosing unit 1 from the assembly port 14, or may remain in a liquid state in the aerosol space S to wait to be nebulized again). However, the present disclosure is not limited thereto. The aerosol outlet 13 may alternatively be provided on a side surface or a bottom surface of the enclosing unit 1, and a flow direction of the condensed droplet is additionally designed to be different from the flow direction of the aerosol passing through the aerosol outlet 13.

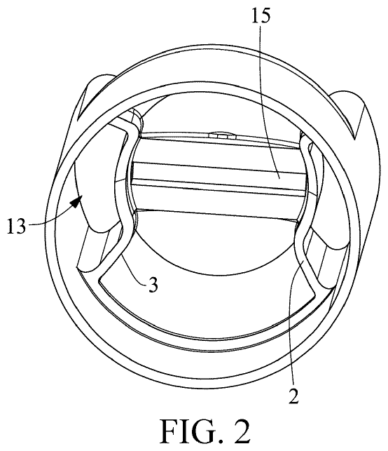

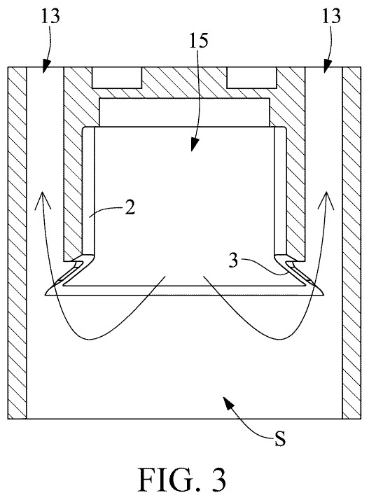

Further, as shown in FIG. 2 and FIG. 3, in this embodiment, the aerosol size control core further includes a particle size screening structure 2. The particle size screening structure 2 is disposed in the aerosol space S. The particle size screening structure 2 is configured to prevent an aerosol with a particle size larger than the preset particle size range from passing through the aerosol outlet 13. The particle size screening structure 2 includes, for example, a flow guiding surface, a baffle, or a micro-mesh. Any structure that can allow an aerosol having a small particle size to pass through and block an aerosol having a large particle size from passing through the aerosol outlet 13 may be used as the particle size screening structure 2 of the present disclosure.

In this embodiment, the particle size screening structure 2 is the flow guiding surface. Only an aerosol with a particle size is sufficiently small can bypass the particle size screening structure 2, and exit the enclosing unit 1 by passing upward through the aerosol outlet 13. The aerosol that has a large particle size and cannot bypass the particle size screening structure 2 still remains inside the enclosing unit 1. Therefore, the particle size screening structure 2 filters or blocks and retains the aerosol with a particle size larger than the preset particle size range, to improve particle size uniformity and efficiency of an output aerosol. In addition, the preset particle size range may be adjusted by adjusting a length, a shape, an opening size, or the like of the particle size screening structure 2, to further limit the particle size of the aerosol output by the aerosol size control core.

Further, as shown in FIG. 3, in this embodiment, the aerosol size control core further includes a liquid guiding portion 3, disposed in the enclosing unit 1. The liquid guiding portion 3 includes at least one inclined surface, groove, convex rib, or curved surface, or any combination thereof. The liquid guiding portion 3 is configured to cause the aerosol (which is the droplet) condensed in the aerosol space S to flow back to the liquid supply area through gravity, inertia, or the like for re-nebulization. The liquid guiding portion 3 may be a structure combined with the particle size screening structure 2, that is, a structure formed by the particle size screening structure 2 and the liquid guiding portion 3 serves dual purposes of “allowing the aerosol with a particle size falls within the preset particle size range to pass through the aerosol outlet 13, and filtering, blocking, and retaining the aerosol with a particle size larger than the preset particle size range”, and “causing the droplet condensed in the aerosol space S to flow back”.

Further, as shown in FIG. 2 and FIG. 3, in this embodiment, the enclosing unit 1 has a plurality of aerosol outlets 13, and a quantity of the particle size screening structures 2 and the liquid guiding portions 3 is the same as a quantity of the plurality of aerosol outlets 13, to cause the plurality of aerosol outlets 13, the particle size screening structures 2, and the liquid guiding portions 3 to be configured in one-to-one correspondence. In other words, a group of the particle size screening structure 2 and the liquid guiding portion 3 are correspondingly disposed in each aerosol outlet 13, to form a plurality of groups of mutually independent and parallel nebulization paths, thereby improving overall nebulization efficiency and aerosol output stability.

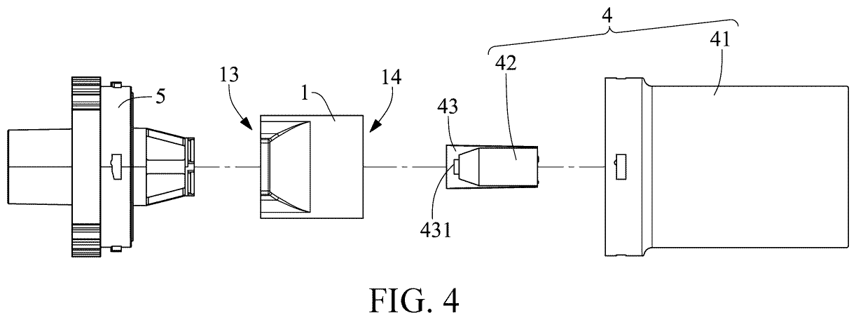

Further, as shown in FIG. 4, in this embodiment, the aerosol size control core is combined with the nebulization unit 4, so that an aerosol size control nebulization device can be formed.

The nebulization unit 4 is configured to nebulize a liquid to generate an aerosol. The aerosol space S accommodates the nebulization unit 4 or is adjacent to the nebulization unit 4, to enclose the aerosol generated by the nebulization unit 4. For example, the nebulization unit 4 is one of an aerodynamic jet nebulization module, an ultrasonic nebulization module, or a micro-mesh nebulization module. A type of the nebulization unit 4 is not limited in the present disclosure.

The nebulization unit 4 shown in FIG. 4 uses the aerodynamic jet nebulization module as an example. The nebulization unit 4 in a form of the aerodynamic jet nebulization module includes a medicine cup 41, a venturi tube 42, and an impact element 43. The venturi tube 42 and a gas nozzle outlet 431 of the venturi tube are components that provide a driving force for a high-speed gas flow impact. The impact element 43 may be formed in various manners. The impact element 43 in this embodiment is fixedly formed at one end of the venturi tube 42, and is then assembled inside the enclosing unit 1 in an assembly manner. However, the present disclosure is not limited thereto. The impact element 43 may alternatively be directly formed on an inner side of the enclosing unit 1, or the impact element 43 is an independent element, and is connected to the enclosing unit 1 and the venturi tube 42 through assembly. The assembly manner, a forming manner, and a specific shape and size of the impact element 43 may be properly changed based on a requirement, and the present disclosure is not limited to implementations related to the impact element and an impact surface.

After a liquid that is entrained by a gas jetted from the nozzle outlet 431 impacts the impact element 43 located inside the enclosing unit 1, droplets of a number of sizes are generated. Only a droplet having a sufficiently small particle size can bypass the particle size screening structure 2, and exit the aerosol size control nebulization device of the present disclosure by passing upward through the aerosol outlet 13. In other words, the impact element 43 is constructed to: generate a primary aerosol by being impacted by a high-speed airflow generated by the nebulization unit 4.

Further, in this embodiment, the aerosol size control nebulization device further includes an output tube 5. The output tube 5 is in communication with the aerosol outlet 13, to direct the aerosol within the preset particle size range that is directed out of the aerosol outlet 13 to the human body.

Further, as shown in FIG. 2 and FIG. 3, in this embodiment, the enclosing unit 1 further has a mounting portion 15, and is configured to be aligned with the impact element 43 of the nebulization unit 4. The impact element 43 is disposed in the enclosing unit 1 in a manner of a detachable structure or a fixed structure through the mounting portion 15. In this embodiment, the mounting portion 15 is, for example, a groove (which is a mounting base) with a shape matches the impact element 43. In other embodiments, the mounting portion 15 may alternatively be, for example, a mounting hole, to correspondingly mount the impact element 43.

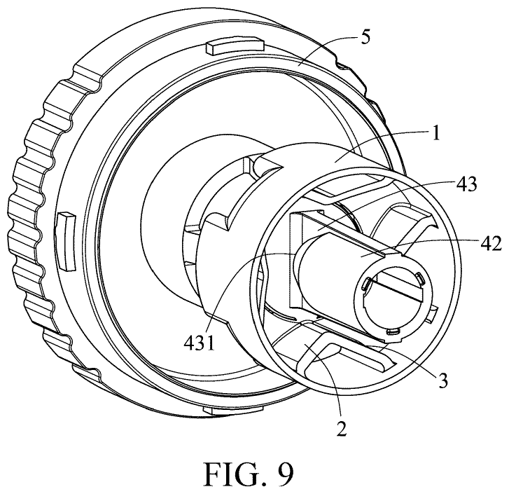

Further, as shown in FIG. 9, in a sixth embodiment of the present disclosure, the nozzle outlet 431 of the nebulization unit 4 and the enclosing unit 1 are of an integrally formed structure, so that a relative position of the nozzle outlet 431 and the enclosing unit 1 remains fixed. Therefore, there is no relative displacement between the nozzle outlet and the enclosing unit, which can improve operation stability of the device and consistency of nebulization quality.

The nebulization device of the present disclosure uses the aerosol size control core to match the aerodynamic jet nebulization module, so that the output aerosol has a mass median aerodynamic diameter (MMAD) of less than 3 μm. In other words, a half of a mass of the aerosol is less than 3 μm. Such an aerosol may enable most aerosol particles to penetrate deep into a respiratory tract and reach the lung, rather than only stay in an oral cavity and an upper respiratory tract of the human body. Therefore, more effective nebulization treatment can be provided, and a burden of inhaling excessive medication on the human body can be reduced.

In the aerosol size control core of the present disclosure, the primary aerosol is screened in the enclosing unit 1. Through the structural design of the enclosing unit 1, the primary aerosol is divided into two parts in the enclosing unit 1. A part of the primary aerosol of a non-preset particle size flows back to the liquid supply area, and an other part of the primary aerosol of a preset particle size exits the enclosing unit 1 through the aerosol outlet 13. Through the foregoing mechanism, the condensed droplet is not adhered to another place than the enclosing unit 1, which causes that the liquid cannot flow back for re-nebulization (which reduces a drug delivery rate). In addition, through the structural design in the enclosing unit 1, a particle size and particle size distribution uniformity of the nebulized aerosol may be controlled, to improve problems of an existing nebulization device in the related art, such as overly broad particle size distribution of the nebulized aerosol, excessive liquid residue, and intermittent and unstable nebulization.

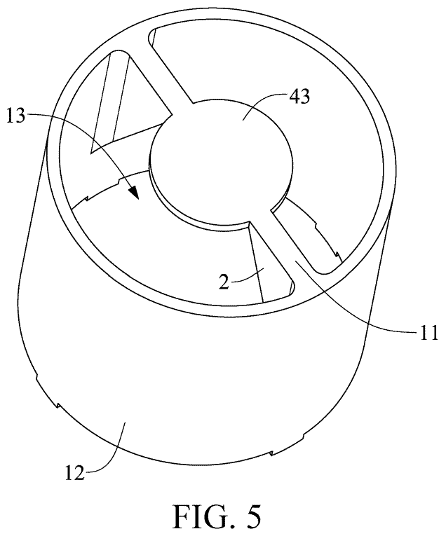

Further, as shown in FIG. 5, the present disclosure provides an aerosol size control core according to a second embodiment. An impact element 43 is directly formed on a top wall 11. A remaining part is hollowed out to form an aerosol outlet 13.

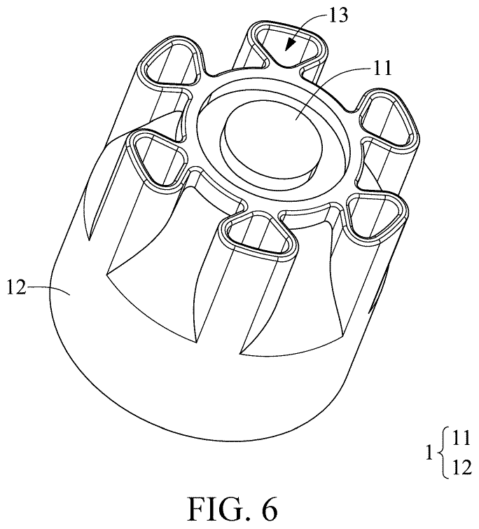

Further, as shown in FIG. 6, the present disclosure provides an aerosol size control core according to a third embodiment. A main difference between the aerosol size control core according to the third embodiment and the aerosol size control core according to the first embodiment lies in a shape and a quantity of aerosol outlets 13, and patterns of a particle size screening structure 2 and a liquid guiding portion 3. In the third embodiment, the quantity of the aerosol outlets 13 is six. The aerosol outlets 13 according to the third embodiment each have a group of the particle size screening structure 2 and the liquid guiding portion 3.

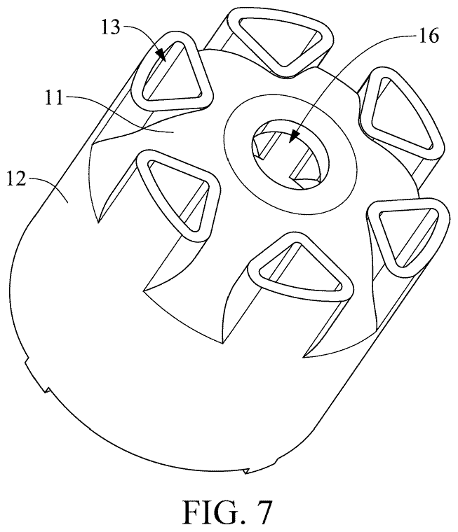

Further, as shown in FIG. 7, the present disclosure provides an aerosol size control core according to a fourth embodiment. A main difference between the aerosol size control core according to the fourth embodiment and the aerosol size control cores according to the foregoing embodiments lies in that an enclosing unit 1 has a through hole 16, and each of aerosol outlets 13 surrounds the through hole 16. A purpose of the through hole 16 is to mount a bottle body and an impact element (which are external elements) that are combined with the existing nebulization device, to generate a nebulized droplet.

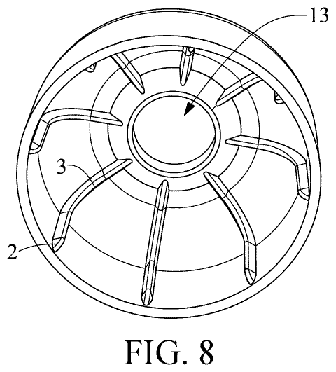

Further, as shown in FIG. 8, the present disclosure provides an aerosol size control core according to a fifth embodiment. A main difference between the aerosol size control core according to the fifth embodiment and the aerosol size control cores according to the foregoing embodiments lies in that the aerosol size control core according to the fifth embodiment only has a single aerosol outlet 13, and the aerosol outlet 13 further has a function of the foregoing through hole 16. In other words, the aerosol size control core according to the fifth embodiment is also configured to be directly combined with the existing nebulization device, and an impact element (which is an external element) is directly disposed in the single aerosol outlet 13. After bypassing a particle size screening structure 2, an aerosol generated by the impact element passes through the aerosol outlet 13, so that particle size screening and control can also be achieved. In the fifth embodiment, a plurality of groups of particle size screening structures 2 and liquid guiding portions 3 are commonly used for the single aerosol outlet 13.

The foregoing embodiments show that the aerosol size control core and the aerosol size control nebulization device of the present disclosure can present diversified structures. The impact element 43 may be an internal element, or may be combined with the existing nebulization device product (which is the external element). The impact surface of the impact element 43 may be designed in different shapes, such as a flat surface, a concave surface, or a crossbar, to generate the aerosol. By designing the size, shape, quantity, and positioning of the aerosol outlet 13, the particle size, the particle size distribution uniformity, and a nebulization rate of the aerosol may be controlled, to determine the preset particle size range.

### Experimental Verification

To verify that the aerosol size control core and the aerosol size control nebulization device of the present disclosure are indeed better than a number of existing nebulization devices, and the present disclosure provides the following test report. Effectiveness of a commercial small volume jet nebulization device is evaluated in terms of aspects such as particle size distribution of an aerosol after a high-pressure gas is nebulized, a nebulization speed, and residual medication in a medicine cup.

### Test Items:

1. The following small volume jet nebulization devices were tested.
A. A small volume jet nebulization device of a company G
B. A small volume jet nebulization device of a company P
C. The aerosol size control nebulization device of the present disclosure
2. A particle size of a nebulization device was tested based on Annex D.
Test basis: Annex D (which is a test method for particle sizing) of an ISO 27427:2013 standard
Research conditions: According to the ISO standard, a room temperature was maintained at 23±2° C., and relative humidity was 45% to 75%.
Research equipment and settings: According to the Annex D standard, a Next Generation Impactor (MSP, Shoreview, Minnesota) was used. An operation flow rate was calibrated by using a dry gas flowmeter (TSI Instrument Inc. Shoreview, Minnesota, Model 4040), and was set at 15.0±0.1 L/min. A collection layer was divided into 8 layers. After a calibration experiment, cut-off particle sizes of collected aerosols were respectively 14.10 μm, 8.61 μm, 5.39 μm, 3.30 μm, 2.08 μm, 1.36 μm, 0.98 μm, and less than 0.5 μm.
Reagent water: Secondarily filtered water for research
Test solution: 0.1% salbutamol sulfate (Taiwan Biotech Co., Ltd.)
Drug dosage analysis: A sample absorbance value was detected by using a UV-Vis spectrophotometer (ThermoScitific Inc., Waltham, MA USA) at a wavelength of 276 nm. Before an experiment, a solution with a known concentration was prepared, after the solution was put into the spectrophotometer, the absorbance value is obtained, and a related calculation formula was obtained, to calculate a subsequent sample concentration.

### Test Procedure:

A. A suction flow rate of an impact sampler was confirmed to be 15.0±10% L/min by using a TSI flowmeter.
B. 2.5 mL (5 mg) of 0.2% salbutamol solution was drawn up by using a micropipette, and the drug was put into a medicine cup.
C. A set flow rate for 50 psig oxygen was calibrated/confirmed by using the TSI flowmeter.
D. Oxygen was set to a specified flow rate, to start nebulization.
E. Nebulization was performed until no aerosol was generated, and nebulization time was recorded.
F. An impactor was disassembled, secondary water was added to each layer for dissolution, and the drug was dissolved and extracted by shaking the solution for two minutes.
G. The extracted drug was measured by using a spectrophotometer to determine an absorbance value of the liquid medicine, and a drug dosage was calculated by using an “absorbance value-concentration” formula.
H. Mass data of particles deposited at each level was input into a dedicated program, to calculate deposited drug dosage distribution, to obtain an MMAD, a geometric standard deviation (GSD), and a proportion of inhalable particles.

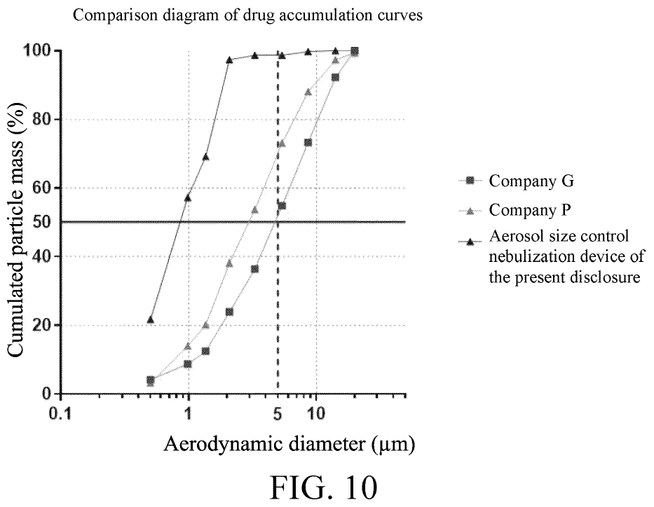

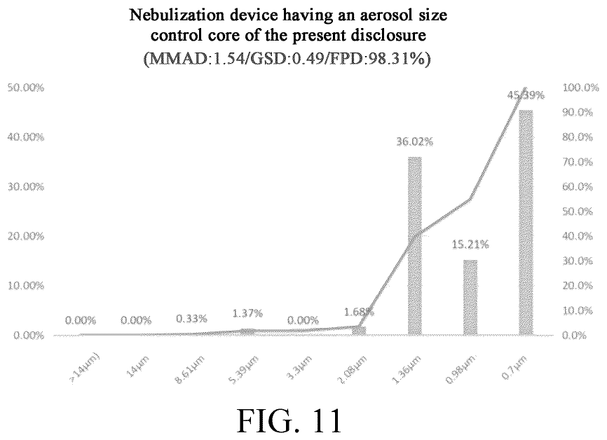

A test result is shown in FIG. 10. FIG. 10 is a comparison diagram of drug accumulation curves of a small volume jet nebulization device of a company G, a small volume jet nebulization device of a company P, and a nebulization device having an aerosol size control core of the present disclosure. FIG. 11 is a drug particle size distribution diagram of a nebulization device using an aerosol size control core according to the present disclosure.

It can be learned from the test result that a particle size of a droplet produced by the nebulization device using the aerosol size control core of the present disclosure is obviously smaller than that of a droplet produced by a control group (the small volume jet nebulization device of the science and technology company G and the small volume jet nebulization device of the company P). It is worth mentioning that the particle size of more than 96% of the aerosol generated by the nebulization device using the aerosol size control core of the present disclosure is less than or equal to 1.36 μm. The foregoing test result shows that the aerosol size control core of the present disclosure has a significant effect on reducing the particle size of the aerosol (which is reduced from original 5.03 μm to 1.54 μm) and increasing a quantity of aerosols having a smallest particle size.

The present disclosure has been disclosed with preferred embodiments above. However, a person skilled in the art should understand that the embodiments are only used to describe the present disclosure, and should not be construed as limiting the scope of the present disclosure. It should be noted that any equivalent change or replacement to the embodiments should be set to fall within the scope of the present disclosure. Therefore, the protection scope of the present disclosure shall be subject to the content scope defined by the claims.

1. An aerosol size control core, configured to match a nebulization unit, and comprising:
an enclosing unit, configured to enclose an aerosol generated by the nebulization unit, wherein the enclosing unit defines an internal aerosol space, to form a controlled flow environment;
wherein the enclosing unit has at least one aerosol outlet, the at least one aerosol outlet is in communication with the aerosol space, and the at least one aerosol outlet is constructed to direct an aerosol within a preset particle size range in the aerosol space out of the enclosing unit; and
the enclosing unit is constructed to: cause a flow direction of a droplet formed by aerosol condensation in the aerosol space to be different from a flow direction of the aerosol directed out of the enclosing unit through the at least one aerosol outlet.

2. The aerosol size control core according to claim 1, wherein the at least one aerosol outlet is provided on a top surface, a side surface, or a bottom surface of the enclosing unit.

3. The aerosol size control core according to claim 1, further comprising a particle size screening structure, wherein the particle size screening structure is disposed in the aerosol space, and the particle size screening structure is configured to prevent an aerosol with a particle size larger than the preset particle size range from passing through the at least one aerosol outlet.

4. The aerosol size control core according to claim 3, wherein the particle size screening structure comprises one of a flow guiding surface, a baffle, or a micro-mesh.

5. The aerosol size control core according to claim 4, further comprising a liquid guiding portion disposed in the enclosing unit, wherein the liquid guiding portion comprises at least one inclined surface, groove, convex rib, or curved surface, or any combination thereof, and the liquid guiding portion is configured to cause the aerosol condensed in the aerosol space to flow back to a liquid supply area.

6. The aerosol size control core according to claim 5, wherein the enclosing unit has a plurality of aerosol outlets, and a quantity of the particle size screening structures and the liquid guiding portions is the same as a quantity of the plurality of aerosol outlets, to cause the plurality of aerosol outlets, the particle size screening structures, and the liquid guiding portions to be configured in one-to-one correspondence.

7. An aerosol size control nebulization device, comprising:
a nebulization unit, configured to nebulize a liquid to generate an aerosol; and
the aerosol size control core according to claim 1, wherein the aerosol space accommodates the nebulization unit or is adjacent to the nebulization unit, to enclose the aerosol generated by the nebulization unit.

8. The aerosol size control nebulization device according to claim 7, wherein the nebulization unit is one of an aerodynamic jet nebulization module, an ultrasonic nebulization module, or a micro-mesh nebulization module.

9. The aerosol size control nebulization device according to claim 8, wherein the nebulization unit is the aerodynamic jet nebulization module, an impact element is disposed inside the enclosing unit, and the impact element is constructed to: generate a primary aerosol by being impacted by a high-speed airflow generated by the nebulization unit.

10. The aerosol size control nebulization device according to claim 9, wherein the enclosing unit further has a mounting portion, and is configured to be aligned with a nozzle of the nebulization unit, and the impact element is disposed in the enclosing unit by using a detachable structure or a fixed structure through the mounting portion.

11. The aerosol size control nebulization device according to claim 8, wherein a nozzle of the nebulization unit and the enclosing unit are of an integrally formed structure, and a relative position of the nozzle and the enclosing unit remains fixed.
